(19) United States
(12) Patent Application Publication
(10) Pub. No.: US 2026/0191762 A1
(43) Pub. Date: Jul. 9, 2026

(54) LIP COSMETIC

(71) Applicant: SHISEIDO COMPANY, LTD., Chuo-ku, Tokyo (JP)

(72) Inventors: Akari MIZUGUCHI, Tokyo (JP); Noriko TOMITA, Tokyo (JP)

(73) Assignee: SHISEIDO COMPANY, LTD., Chuo-ku, Tokyo (JP)

(21) Appl. No.: 19/131,632

(22) PCT Filed: Dec. 8, 2023
(86) PCT No.: PCT/JP2023/043956

§ 371 Date: May 21, 2025

(30) Foreign Application Priority Data
Dec. 23, 2022 (JP) 2022-207591

(51) Int. Cl. A61K 8/92; A61K 8/02; A61K 8/37; A61K 8/891; A61Q 1/04

(52) U.S. Cl. CPC A61K 8/922; A61K 8/0216; A61K 8/37; A61K 8/891; A61Q 1/04; A61K 2800/43; A61K 2800/612; A61K 2800/651

(57) ABSTRACT

[Problem] An objective of the present invention is to provide a lip cosmetic that has excellent pigment dispersibility and that is vividly colorful in appearance. [Solution] The lip cosmetic of the present invention is characterized by containing (A) sunflower wax; (B) a pigment surface-treated with one or more types of substances selected from among metal soaps and amino acids or salts thereof; and (C) a non-volatile liquid non-polar oil; wherein a blending percentage of the component (C) with respect to a total amount of liquid oils in the cosmetic is 1% to 37.5% by mass.

### TECHNICAL FIELD

The present invention relates to a lip cosmetic that has excellent pigment dispersibility and that is vividly colorful in appearance.

### BACKGROUND ART

In lipstick cosmetics such as rouge, lipstick and lip gloss, solid oils such as waxes and tallows are generally blended in order to obtain good fill-moldability at the time of manufacture.

During the process of research by the inventors, there was a problem in that, when manufacturing lipstick cosmetics, in the case in which sunflower wax was used as a solid oil, if a pigment with hydrophobized surfaces was blended, the pigment would not sufficiently disperse and the cosmetic would have a dull appearance.

As solid cosmetics in which sunflower wax is blended, there is art for obtaining an oil-based solid cosmetic having excellent properties in use, texture when applied and stability due to combining sunflower seed wax, inulin fatty acid ester and a volatile oil (Patent Document 1), and art for obtaining an oil-based solid lip cosmetic having excellent compatibility with skin, sense of penetration into the lips and moldability due to combining sunflower wax, a liquid branched higher alcohol having a total of 20-24 carbon atoms and a liquid ester oil having a specific viscosity (Patent Document 2).

However, with the art in Patent Document 1 and Patent Document 2, the obtained cosmetics could not be considered to be sufficiently colorful in appearance.

### RELATED ART

### Patent Documents

Patent Document 1: JP 5895248 B
Patent Document 2: JP 6826807 B

### SUMMARY OF THE INVENTION

### Problem to be Solved by the Invention

An objective of the present invention is to provide a lip cosmetic that has excellent pigment dispersibility and that is vividly colorful in appearance.

As a result of performing diligent investigations towards solving the aforementioned problem, the inventors discovered that the pigment dispersibility is improved and the appearance is made vividly colorful by blending a pigment that has been surface-treated with one or more types of substances selected from among metal soaps and amino acids or salts thereof, and a certain amount of a non-volatile liquid non-polar oil into a lip cosmetic in which sunflower wax has been blended, thereby completing the present invention.

That is, the present invention provides a lip cosmetic characterized by containing:
(A) sunflower wax;
(B) a pigment surface-treated with one or more types of substances selected from among metal soaps and amino acids or salts thereof; and
(C) a non-volatile liquid non-polar oil;
wherein a blending percentage of the component (C) with respect to a total amount of liquid oils in the cosmetic is 1% to 37.5% by mass.

### Effects of the Invention

By having the above-mentioned features, the present invention can obtain a lip cosmetic with improved dispersibility of the pigment blended into the cosmetic and with a vividly colorful appearance.

### MODES FOR CARRYING OUT THE INVENTION

The cosmetic according to the present invention is characterized by containing (A) sunflower wax, (B) a pigment surface-treated with one or more types of substances selected from among metal soaps and amino acids or salts thereof, and (C) a non-volatile liquid non-polar oil. Hereinafter, the respective components constituting the lip cosmetic of the present invention will be explained in detail.

### <(A) Sunflower Wax>

The (A) sunflower wax (hereinafter sometimes referred to simply as “component (A)”) in the present invention is not particularly limited as long as it can generally be blended into cosmetics. Sunflower wax is a vegetable wax that is obtained when sunflower seed oil is dewaxed, that contains a long-chain saturated fatty acid ester constituted by a long-chain alcohol and a long-chain fatty acid as the main component, and that is a solid oil with a melting point in the range of 70 to 85° C. The INCI (International Nomenclature of Cosmetic Ingredients) name is Helianthus Annuus (Sunflower) Seed Wax (cosmetic product indication name: sunflower seed wax), and commercial products include purified sunflower wax (manufactured by Yokozeki Oil & Fat Industries Co., Ltd.), etc.

The blended amount of component (A) is not particularly limited as long as it is an amount that can normally be blended into a lip cosmetic. For example, the lower limit value of the blended amount may be 0.5% by mass, 1% by mass, 3% by mass, 5% by mass, etc., and the upper limit value may be 35% by mass, 30% by mass, 25% by mass, 20% by mass, etc with respect to the overall amount of the cosmetic. Thus, the blended amount range may be 0.5% to 35% by mass, 1% to 30% by mass, etc.

### <(B) Pigment>

The (B) pigment surface-treated with one or more types of substances selected from among metal soaps and amino acids or salts thereof (hereinafter sometimes referred to simply as “component (B)”) in the present invention refers to an inorganic pigment surface-treated with a hydrophobic surface treatment agent containing one or more types of substances selected from among metal soaps and amino acids or salts thereof.

Examples of the “amino acids” used as the surface treatment agent in component (B) include glutamic acid, aspartic acid, lysine, etc., among which glutamic acid and aspartic acid are preferable. Additionally, the “amino acids” may be “amino acids acylated by a saturated fatty acid”, in which acyl groups, preferably saturated fatty acids with 12 to 20 carbon atoms, are condensed on the amino groups in the amino acids. Examples of “acyl groups” include stearoyl groups, lauroyl groups, etc. The “salts” may be selected from among alkali metal salts such as sodium or potassium, alkaline earth metal salts, etc., among which sodium salts are preferable. Specific examples of acylated amino acids and salts thereof include disodium stearoyl glutamate, sodium lauroyl glutamate, sodium lauroyl aspartate and lauroyl lysine.

The “metal soaps” used as the surface treatment agent in component (B) are metal salts of saturated or unsaturated higher fatty acids and are not particularly limited, but are preferably salts, such as aluminum, calcium, magnesium and zinc salts, of saturated and/or unsaturated fatty acids having 8 to 24 carbon atoms, particularly 12 to 18 carbon atoms. Specific examples include aluminum stearate, magnesium isostearate, aluminum dimyristate, etc.

Component (B) in the present invention is preferably a pigment treated with a surface treatment agent containing an amino acid or a salt thereof, and is more preferably a pigment surface-treated with a surface treatment agent containing disodium stearoyl glutamate.

As the surface treatment agent of component (B) in the present invention, just one or more types of substances selected from among the above-mentioned metal soaps and amino acids or salts thereof may be used, or other compounds that are generally used as surface treatment agents for powders may be further included in addition to the above-mentioned one or more types of substances selected from among metal soaps and amino acids or salts thereof.

Specific examples of pigments include red iron oxide (Bengal red), iron titanate, Y-iron oxide, yellow iron oxide, ocher, black iron oxide, carbon black, low-order titanium oxide, mango violet, cobalt violet, chromium oxide, chromium hydroxide, cobalt titanate, ultramarine blue, Prussian blue, etc. Among these, it is preferable to use a pigment-grade iron oxide such as yellow iron oxide, red iron oxide or black iron oxide, a pigment-grade titanium oxide, etc. as the pigment in the present invention. In this case, pigment grade means having an average particle diameter of 100 nm to 1 μm.

The blended amount of component (B) is not particularly limited as long as a desired color can be obtained. For example, the lower limit value of the blended amount may be 0.5% by mass, 1% by mass, etc., and the upper limit value may be 30% by mass, 25% by mass, 20% by mass, 15% by mass, etc. with respect to the overall amount of the cosmetic. Thus, the blended amount range may be 0.5% to 30% by mass, 1% to 25% by mass, etc. If the blended amount is less than 0.5% by mass, sufficient color cannot be obtained, and if 30% by mass is exceeded, the stability tends to become poor.

### <(C) Non-Volatile Liquid Non-Polar Oil>

The (C) non-volatile liquid non-polar oil (hereinafter sometimes referred to simply as “component (C)”) in the present invention refers to an oil constituted by hydrocarbons, which does not exhibit volatility at ambient temperature (25° C.) and ambient pressure (1 atm (9.8×104 Pa)) (for example, oils having a boiling point of approximately 200° C. or higher at ambient pressure are included), and which is fluid at ambient temperature and ambient pressure.

Examples of component (C) in the present invention include hydrocarbon oils such as mineral oil, squalane, squalene, pristane, paraffin, isoparaffin, isohexadecane, hydrogenated polyisobutene, olefin oligomers, etc.

Component (C) is blended at a percentage of 1% to 37.5% by mass with respect to the total amount of the liquid oils in the cosmetic. The blending percentage of component (C) is the blending percentage with respect to the total amount of liquid oils and semi-solid oils when a semi-solid oil is blended into the cosmetic. The blended amount of component (C) is not limited as long as this percentage is satisfied.

As examples of the blended amount of component (C), 0.8% to 35% by mass with respect to the overall amount of the cosmetic is preferable, and 1% to 30% by mass is more preferable. If the blended amount is too small or too large, there are cases in which the cosmetic lacks vividness in appearance.

The “semi-solid oil” in the present specification refers to an oil which does not exhibit fluidity at ambient temperature (25° C.), and which has a melting point of 30 to 60° C., preferably a melting point of 30 to 45° C., at 1 atm. A “liquid oil” refers to an oil that exhibits fluidity at ambient temperature (25° C.).

Examples of liquid oils other than component (C) that can be blended into the cosmetic of the present invention include silicone oils, ester oils, liquid oils/fats, oil-soluble UV absorbing agents, semi-solid oils, etc.

Silicone oils include linear silicone oils such as dimethyl polysiloxane (also known as dimethicone), methylphenyl polysiloxane, diphenyl polysiloxane, methylhydrogen polysiloxane and diphenylsiloxy phenyl trimethicone, and cyclic silicone oils such as decamethyl cyclopentasiloxane, dodecamethyl cyclohexasiloxane and octamethyl cyclotetrasiloxane.

Ester oils include isodecyl isostearate, ethylhexyl palmitate, glyceryl diisostearate, isopropyl isostearate, sucrose tetraisostearate, triisostearin, tripropylene glycol dineopentanoate, isononyl isononanoate, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, cetyl ethylhexanoate, ethylene glycol di-2-ethylhexanoate, dipentaerythritol fatty acid ester, N-alkyl glycol monoisostearate, neopentyl glycol dicaprate, diisostearyl malate, glycerin di-2-heptylundecanoate, trimethylolpropane tri-2-ethylhexanoate, polyglyceryl-2 triisostearate, trimethylolpropane triisostearate, pentaerythrityl tetra-2-ethylhexanoate, triethylhexanoin (also known as glyceryl tri-2-ethylhexanoate), glycerin trioctanoate, glycerin triisopalmitate, cetyl 2-ethylhexanoate, 2-ethylhexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, etc.

Liquid oils/fats include palm oil, linseed oil, camellia oil, macadamia nut oil (macadamia seed oil), corn oil, olive oil, avocado oil, sasanqua oil, castor oil, safflower oil, persic oil, cinnamon oil, jojoba oil, grape oil, almond oil, rapeseed oil, sesame oil, sunflower oil, wheat germ oil, rice germ oil, rice bran oil, cottonseed oil, soybean oil, peanut oil, tea seed oil, evening primrose oil, egg yolk oil, liver oil, etc.

Oil-soluble UV absorbing agents include ethylhexyl methoxycinnamate, octocrylene, dimethicodiethyl benzalmalonate, polysilicone-15, t-butyl methoxydibenzoylmethane, ethylhexyl triazone, diethylamino hydroxybenzoyl hexyl benzoate, bis-ethylhexyloxyphenol methoxyphenyl triazine, oxybenzone-3, methylene bis-benzotriazolyl tetramethylbutylphenol, homosalate, ethylhexyl salicylate, drometrizole trisiloxane, etc.

Semi-solid oils include vaseline, pentaerythrityl tetra(behenate/benzoate/ethylhexanoate), dipentaerythrityl hexahydroxystearate, phytosteryl macadamiate, dimer dilinoleic acid ester, macadamia nut oil polyglyceryl-6 esters behenate, dipentaerythrityl tetra(hydroxystearate/isostearate), bis-diglyceryl polyacyladipate-2, etc.

The blended amount of the liquid oils (including semi-solid oils) other than component (C) is not particularly limited as long as it is an amount that is normally blended into lip cosmetics. Examples include 30% to 95% by mass, 50% to 90% by mass, etc.

In the cosmetic of the present invention, an oil phase thickener (gelling agent) that is normally blended in order to adjust cosmetics to be in solid stick form or in paste form may be blended in addition to the above-mentioned components (A) to (C) and the liquid oils other than component (C).

Examples of oil phase thickeners (gelling agents) include dextrin fatty acid ester, glycerin fatty acid ester, organically modified clay minerals, etc. Dextrin fatty acid esters include, for example, dextrin myristate, dextrin palmitate, dextrin (palmitate/2-ethylhexanoate), etc. Glycerin fatty acid esters include, for example, glyceryl behenate, glyceryl octastearate, glyceryl eicosanoate, etc.

The blended amount of the oil phase thickener in a solid or paste-type cosmetic is 2.5% by mass to 15% by mass, preferably 3.0% by mass to 12% by mass, or 4.0% by mass to 10% by mass.

The cosmetic of the present invention may include other arbitrary components that can normally be blended into lip cosmetics within a range not hindering the effects of the present invention. The arbitrary components include, but are not limited to, water-soluble polymers, oil-soluble polymers, lower alcohols, higher alcohols, surfactants, powders other than pigments, colorants, dyes, medicinal agents, etc.

The lip cosmetic of the present invention may be prepared as a water-free oil-based cosmetic, or may be prepared as an oil-based cosmetic containing a small amount (for example, 1.5% by mass or less) of water. As specific forms, forms such as solid (for example, sticks), pastes, creams and gels are possible, with the lip cosmetic being particularly suited to be in solid stick or paste form.

### EXAMPLES

The present invention will be described in further detail by providing examples below. However, the present invention is not limited, in any way, by these examples. The blended amounts, where not particularly noted, are indicated in percentage by mass with respect to the system in which the relevant component is blended. Before specifically explaining each example, the evaluation methods that were employed will be explained.

### 1. Evaluation of Dispersibility by Colorimetry

After preparing each sample, a measurement container was filled with the sample and the brightness difference of each sample at room temperature was measured by using a Ci7600 (manufactured by X-Rite, Inc.), which is a common integrating sphere colorimeter. The brightness of each sample measured with respect to a standard sample (STD) is indicated in the table.

In general, the smaller the primary particle diameter of a pigment is, the whiter the surface reflection will be, making the the brightness greater. In the present evaluation, it is assessed that, when a pigment is uniformly dispersed and the particle diameter is kept small, there is more white light due to surface reflection and the brightness increases, and when the pigment in a sample aggregates or the like and the particle diameter becomes large, more light is absorbed and the brightness decreases. Thus, in the present measurement results, the dispersion state can be evaluated to be better when the brightness is higher.

When the brightness in the table is a positive value relative to the standard sample (STD), this means that the dispersion state of the measured sample is better than that of the standard sample, and the appearance is bright. When the brightness is a negative value, this means that the dispersion state of the measured sample is worse than that of the standard sample, and the appearance is dark. The appearance being dark means having a dull appearance.

### Example 1 and Comparative Examples 1 and 2

Rouges (solid lip cosmetics) were prepared with the formulations indicated in Table 1 below. The brightnesses of Comparative Examples 1 and 2 were measured by using, for reference, the value of the cosmetic of Example 1, measured by the aforementioned method, as a sample of the prepared rouge. The results are indicated in the table.

The “amino acid-treated yellow iron oxide” in Tables 1-5 below is a yellow iron oxide surface-treated with a hydrophobic treatment agent containing disodium stearoyl glutamate and isostearyl sebacate.

TABLE 1

| | | | Comp. | Comp. |
|---|---|---|---|---|
| Class | Component | Ex. 1 | Ex. 1 | Ex. 2 |
| Component (A) | Sunflower seed wax | 10 | 10 | 10 |
| Component (C) | Squalane | 20 | — | 20 |
| | Diisostearyl malate | 30 | 40 | 30 |
| | Polyglyceryl-2 triisostearate | 20 | 30 | 20 |
| | Diphenyl siloxyphenyl trimethicone | 10 | 10 | 10 |
| Component (B) | Amino acid-treated yellow iron oxide | 10 | 10 | — |
| | Silicone-treated yellow iron oxide | — | — | 10 |
| | Total | 100 | 100 | 100 |
| | Brightness | STD | −0.28 | −1.15 |

As indicated in Table 1, the cosmetics of Comparative Example 1, which did not contain component (C) of the present invention, and of Comparative Example 2, in which yellow iron oxide surface-treated with silicone was blended instead of component (B) of the present invention, both had negative values as the measurement values, thus indicating that the cosmetics of Comparative Examples 1 and 2 were dull-colored in appearance in comparison with the cosmetic of Example 1 according to the present invention.

### Examples 1 to 5 and Comparative Examples 1, 3 and 4

Rouges (solid lip cosmetics) were prepared with the formulations indicated in Table 2 below. The brightnesses of the respective samples were measured by using, for reference, the value of the cosmetic of Comparative Example 1, measured by the aforementioned method, as a sample of the prepared rouge. The results are indicated in the table.

TABLE 2

| | | Comp. | Comp. | Ex. | Ex. | Ex. | Ex. | Ex. | Comp. |
|---|---|---|---|---|---|---|---|---|---|
| Class | Component | Ex. 1 | Ex. 3 | 2 | 1 | 3 | 4 | 5 | Ex. 4 |
| Component | Sunflower seed | 10 | 10 | 10 | 10 | 10 | 10 | 10 | 10 |
| (A) | wax | | | | | | | | |
| Component | Squalane | — | 40 | 30 | 20 | 10 | 5 | 1 | 0.5 |
| (C) | | | | | | | | | |
| | Diisostearyl | 40 | 20 | 25 | 30 | 35 | 35 | 35 | 35 |
| | malate | | | | | | | | |
| | Polyglyceryl-2 | 30 | 10 | 15 | 20 | 25 | 30 | 34 | 34.5 |
| | triisostearate | | | | | | | | |
| | Diphenyl | 10 | 10 | 10 | 10 | 10 | 10 | 10 | 10 |
| | siloxyphenyl | | | | | | | | |
| | trimethicone | | | | | | | | |
| Component | Amino acid- | 10 | 10 | 10 | 10 | 10 | 10 | 10 | 10 |
| (B) | treated yellow | | | | | | | | |
| | iron oxide | | | | | | | | |
| | Total | 100 | 100 | 100 | 100 | 100 | 100 | 100 | 100 |
| | Blended | 0 | 50 | 37.5 | 25 | 12.5 | 6.25 | 1.25 | 0.625 |
| | percentage (%) | | | | | | | | |
| | of component | | | | | | | | |
| | (C) in total | | | | | | | | |
| | amount of liquid | | | | | | | | |
| | oils | | | | | | | | |
| | Brightness | STD | −0.54 | 0.22 | 0.26 | 0.20 | 0.51 | 0.43 | −0.02 |

As indicated in Table 2, the brightnesses of the cosmetics of Examples 1 to 5, in which the blended percentage of component (C) with respect to the total amount of liquid oils and semi-solid oils was 1% to 37.5% by mass, were all positive values, indicating that they were vividly colored in appearance in comparison with the cosmetic of Comparative Example 1, which was the standard sample.

In contrast therewith, the brightnesses of the cosmetics of Comparative Example 3, in which the blended percentage of component (C) was 50% by mass, and of Comparative Example 4, in which the blended percentage of component (C) was 0.625%, both were negative values, thus indicating that they were even more dull-colored in appearance than the cosmetic of Comparative Example 1.

### Examples 6 to 9 and Comparative Examples 5 and 6

Rouges (solid lip cosmetics) were prepared with the formulations indicated in Table 3 below. The brightnesses of the respective samples were measured by using, for reference, the value of the cosmetic of Comparative Example 5, measured by the aforementioned method, as a sample of the prepared rouge. The results are indicated in the table.

TABLE 3

| | | Comp. | Ex. | Ex. | Ex. | Ex. | Comp. |
|---|---|---|---|---|---|---|---|
| Class | Component | Ex. 5 | 6 | 7 | 8 | 9 | Ex. 6 |
| Component | Sunflower | 10 | 10 | 10 | 10 | 10 | 10 |
| (A) | seed wax | | | | | | |
| Component | Squalane | — | 0.89 | 10 | 30 | 33.38 | 35 |
| (C) | | | | | | | |
| | Diisostearyl | 50 | 49.11 | 40 | 20 | 16.62 | 15 |
| | malate | | | | | | |
| | Polyglyceryl-2 | 39 | 39 | 39 | 39 | 39 | 39 |
| | triisostearate | | | | | | |
| Component | Amino acid- | 1 | 1 | 1 | 1 | 1 | 1 |
| (B) | treated yellow | | | | | | |
| | iron oxide | | | | | | |
| | Total | 100 | 100 | 100 | 100 | 100 | 100 |
| | Blended | 0 | 1 | 11.2 | 33.7 | 37.5 | 39.3 |
| | percentage (%) | | | | | | |
| | of component | | | | | | |
| | (C) in total | | | | | | |
| | amount of | | | | | | |
| | liquid oils | | | | | | |
| | Brightness | STD | 0.06 | 0.31 | 0.08 | 0.18 | −0.12 |

The cosmetics of the examples and the comparative examples in Table 3 are cosmetics in which the blended amount of the pigment (B) was lower in comparison with the cosmetics of the examples and comparative examples in Table 2.

As indicated in Table 3, even in cosmetics with a lower blended amount of the pigment (B), the brightnesses of the cosmetics of Examples 6 to 9, in which the blended percentage of component (C) with respect to the total amount of liquid oils and semi-solid oils was 1% to 37.5% by mass, were all positive values, indicating that they were vividly colored in appearance in comparison with the cosmetic of Comparative Example 5, which was the standard sample.

In contrast therewith, the brightness of the cosmetic of Comparative Example 6, which had a lower blended amount of pigment (B) and in which the blended percentage of component (C) was 39.3% by mass, was a negative value, thus indicating that it was even more dull-colored in appearance than the cosmetic of Comparative Example 5.

### Examples 10 to 12 and Comparative Examples 5 and 7

Rouges (solid lip cosmetics) were prepared with the formulations indicated in Table 4 below. The brightnesses of the respective samples were measured by using, for reference, the value of the cosmetic of Comparative Example 5, measured by the aforementioned method, as a sample of the prepared rouge. The results are indicated in the table.

TABLE 4

| | | Comp. | Ex. | Ex. | Ex. | Comp. |
|---|---|---|---|---|---|---|
| Class | Component | Ex. 5 | 10 | 11 | 12 | Ex. 7 |
| Component | Sunflower seed wax | 10 | 10 | 10 | 10 | 10 |
| (A) | | | | | | |
| Component | Olefin oligomer | — | 0.89 | 10 | 30 | 35 |
| (C) | | | | | | |
| | Diisostearyl malate | 50 | 49.11 | 40 | 20 | 15 |
| | Polyglyceryl-2 | 39 | 39 | 39 | 39 | 39 |
| | triisostearate | | | | | |
| Component | Amino acid-treated | 1 | 1 | 1 | 1 | 1 |
| (B) | yellow iron oxide | | | | | |
| | Total | 100 | 100 | 100 | 100 | 100 |
| | Blended | 0 | 1 | 11.2 | 33.7 | 39.3 |
| | percentage (%) of | | | | | |
| | component (C) in total | | | | | |
| | amount of liquid oils | | | | | |
| | Brightness | STD | 0.34 | 1.16 | 1.17 | −0.02 |

Even in the case in which an olefin oligomer was used as component (C) of the present invention, the brightnesses of the cosmetics of Examples 10 to 12, in which the blended percentage of component (C) with respect to the total amount of liquid oils and semi-solid oils was 1% to 37.5% by mass, were all positive values, indicating that they were vividly colored in appearance in comparison with the cosmetic of Comparative Example 5, which was the standard sample.

In contrast therewith, the brightness of the cosmetic of Comparative Example 7, in which the blended percentage of component (C) was 39.3% by mass, was a negative value, thus indicating that it was even more dull-colored in appearance than the cosmetic of Comparative Example 5.

### Examples 13 to 15 and Comparative Examples 8 and 9

Rouges (paste-type lip cosmetics) were prepared with the formulations indicated in Table 5 below. The brightnesses of the respective samples were measured by using, for reference, the value of the cosmetic of Comparative Example 8, measured by the aforementioned method, as a sample of the prepared rouge. The results are indicated in the table.

TABLE 5

| | | Comp. | Ex. | Ex. | Ex. | Comp. |
|---|---|---|---|---|---|---|
| Class | Component | Ex. 8 | 13 | 14 | 15 | Ex. 9 |
| Component | Sunflower seed wax | 5 | 5 | 5 | 5 | 5 |
| (A) | | | | | | |
| Component | Olefin oligomer | — | 0.99 | 10 | 30 | 40 |
| (C) | | | | | | |
| | Diisostearyl malate | 50 | 49.01 | 40 | 20 | 10 |
| | Macadamia seed oil | 44 | 44 | 44 | 44 | 44 |
| Component | Amino acid-treated | 1 | 1 | 1 | 1 | 1 |
| (B) | yellow iron oxide | | | | | |
| | Total | 100 | 100 | 100 | 100 | 100 |
| | Blended | 0 | 1.1 | 10.6 | 31.9 | 42.6 |
| | percentage (%) of | | | | | |
| | component (C) in total | | | | | |
| | amount of liquid oils | | | | | |
| | Brightness | STD | 0.22 | 1.23 | 1.19 | −0.09 |

Even in the case in which the lip cosmetic of the present invention was prepared in paste form, the brightnesses of the cosmetics of Examples 13 to 15, in which the blended percentage of component (C) with respect to the total amount of liquid oils and semi-solid oils was 1% to 37.5% by mass, were all positive values, indicating that they were vividly colored in appearance in comparison with the cosmetic of Comparative Example 8, which was the standard sample.

In contrast therewith, the brightness of the cosmetic of Comparative Example 9, in which the blended percentage of component (C) was 42.6% by mass, was a negative value, thus indicating that it was even more dull-colored in appearance than the cosmetic of Comparative Example 8.

1. A lip cosmetic containing:
(A) sunflower wax;
(B) a pigment surface-treated with one or more types of substances selected from among metal soaps and amino acids or salts thereof; and
(C) a non-volatile liquid non-polar oil;
wherein a blending percentage of the component (C) with respect to a total amount of liquid oils in the cosmetic is 1% to 37.5% by mass.

2. The lip cosmetic according to claim 1, wherein the component (B) is a pigment surface-treated with a surface treatment agent containing an amino acid or a salt thereof.

3. The lip cosmetic according to claim 1, wherein the component (B) is a pigment surface-treated with a surface treatment agent containing disodium stearoyl glutamate.

4. The lip cosmetic according to claim 1, wherein the component (C) is selected from among hydrocarbon oils.

5. The lip cosmetic according to claim 1, wherein the component (C) is squalane.
